(19) United States
(12) Patent Application Publication
(10) Pub. No.: US 2012/0283545 A1
(43) Pub. Date: Nov. 8, 2012

(54) SYSTEMS AND METHODS FOR DEEP VASCULAR ACCESS

(75) Inventors: Karl-Heinz Kuck, Hamburg (DE); Amr Salahieh, Saratoga, CA (US); Hans Valencia, San Jose, CA (US); Claudio Argento, Felton, CA (US); Tom Saul, Moss Beach, CA (US)

(73) Assignee: Shifamed Holdings, LLC

(21) Appl. No.: 13/311,461

(22) Filed: Dec. 5, 2011

Related U.S. Application Data
(60) Provisional application No. 61/419,758, filed on Dec. 3, 2010.

(51) Int. Cl. A61M 25/09; A61B 8/00; A61B 5/05; A61M 25/06

(52) U.S. Cl. 600/409; 604/164.13; 600/424; 604/164.1

(57) ABSTRACT

Percutaneous endovascular delivery of cardiac prosthetic devices is described through deep vascular access. Specifically, minimally intrusive access is achieved at points in the vascular tree having a size not usually achievable with existing tools. Localizing guide wires having visualization markings enable controlled passage through the vascular system.

### CROSS-REFERENCE TO RELATED APPLICATION

This application claims the benefit of U.S. Provisional Application No. 61/419,758 filed Dec. 3, 2010, which application is incorporated herein by reference.

### BACKGROUND

Percutaneous endovascular delivery of devices such as a diagnostic devices, surgical devices, tools, or implants, herein referred to as surgical devices or delivered devices, has become a common means of performing minimally invasive procedures on patients. Such procedures provide for reductions in risks, cost, and time to recovery over more traditional standard surgical and laparoscopic procedures.

Such procedures typically follow a protocol which incorporates the following steps and components. A needle is placed percutaneously to access an intravascular location at the access site. A guide wire is then delivered thru the needle to the target site. The needle is then removed by sliding it proximally off of the proximal end of the guide wire, leaving the guide wire in place. After the removal of the needle, an introducer catheter is tracked down the guide wire into the vessel. The introduction of the introducer catheter may incorporate the use of a dialator to increase the size of the path. The delivered device is then delivered, typically over the guide wire and through the introducer catheter, to the target site. Of particular interest is the delivery of percutaneously delivered heart valves using such a technique.

Proper access points must be used to facilitate such a procedure. A number of the primary characteristics for an appropriate access site are ease of localization from the surface of the body and a vessel size of appropriate diameter to support the devices being delivered. Typically, access via the femoral arteries is acceptable because these structures are easily localized via palpitation, have relatively large diameters, and directly interface with the aortic trunk. However, in some cases, the femoral arteries may not be able to support such procedures. Such as, the size of the femoral or subclavian artery is too small or tortuous, and an alternative site either past any tortuousity or of larger diameter is required. Such conditions can also be the result of anatomical anomalies, frailty of the tissue, stenosis and or calcification at the site or some intervening location, as the result of a medical condition, amongst other reasons. Additionally, the procedure of delivering the device to the deep vessel access site requires the physician to perform the access step while maintaining x-ray imaging. In such a procedure the physician's hands will likely be in the field of view of the fluoroscope during the portion of the procedure when performing the deep vascular access guide wire and introducer placement. The accumulated exposure to the physician's hands in such a situation could have serious long-term consequences. In these situations an alternate means of providing access to vasculature of appropriate diameters which are deeper and more difficult to reach is required. Alternative sites are available but hard to reach as the larger diameter artery portions are deeper in the tissue and cannot easily be palpitated etc. At present, in such situations, more invasive procedures may be required such as apical approaches for heart valve replacement, which do not have the advantages listed above.

Accordingly, it would be a great advantage to provide for a means of accessing locations in the vascular tree which are of appropriate size and have less critical circulatory targets and can support the introduction of surgical devices, but are at present difficult or impossible to access with present day tools.

Moreover, an ideal apparatus would not require a major investment in capital equipment but would still facilitate the percutaneous placement of introducer sheaths, and thereby provide access for the delivery of surgical devices to deep vascular access sites.

### SUMMARY OF INVENTION

One means of providing such deep vascular access is the use of two guide wires and two access points. One of the guide wires is a localizing guide wire delivered via access through a small vessel to the target access site. The localizing guide wire, which is visible by traditional xray means, can be used as a visual target to localize the site where the second guide wire is to be placed. The second or deep vascular access guide wire then becomes the track by which the deep vascular access introducer is placed. The size of the vessel along the localizing guide wire path may be ascertained via traditional methods aiding the physician in localizing an appropriate deep vascular access site. In such a procedure, a specialized guide wire carries markings to delineate specific positions along the guide wire under fluoroscopy that enables the physician to identify discrete target sites where the vessel diameter becomes large enough to support the deliverable device.

### DESCRIPTION OF FIGURES

FIG. 1 is a guide wire incorporating localization elements along a length of the distal end thereof.

FIG. 2 is multiple embodiments of a sheath with a localization element on a guide wire.

FIG. 3 shows a guide wire with an alternate set of localization elements.

FIG. 4 shows a deep access introducer needle and guidewire.

FIG. 5 shows a needle guide fixture.

FIG. 6 shows a needle guide fixture along with an introducer needle and a localizing guide wire.

FIG. 7 shows a bottom view of a needle guide fixture along with an introducer needle and a localizing guide wire.

FIG. 8 shows a properly aligned needle guide fixture, introducer needle and a localizing guide wire as seen through a fluoroscope when the fluoroscope is also properly aligned.

FIG. 9 shows a properly aligned needle guide fixture, introducer needle and a localizing guide wire as seen through a fluoroscope when the fluoroscope is not properly aligned.

FIG. 10 shows an alternate configuration of a needle guide fixture, introducer needle and a localizing guide wire.

FIG. 11 shows a view of an alternate configuration of a needle guide fixture, introducer needle and a localizing guide wire as seen through a fluoroscope when the system and fluoroscope are all properly aligned.

FIG. 12 shows a view of an alternate configuration of a needle guide fixture, introducer needle and a localizing guide wire as seen through a fluoroscope when the system and fluoroscope are not properly aligned.

FIG. 13 shows common major human arteries and possible access points for a localizing guide wire.

FIG. 14 shows an aortic arch.

FIG. 15 shows an aortic arch with a localizing guide wire in place.

FIG. 16 shows an aortic arch with both a localizing guide wire and introducer needle in place.

FIG. 17 shows an aortic arch with an introducer needle in place and a secondary guide wire delivered through the introducer needle and a localizing guide wire partially removed.

FIG. 18 shows an aortic arch with a guide wire in place after complete or partial removal of a introducer needle and a localizing guide wire.

FIG. 19 shows an aortic arch with an introducer catheter partially tracked down a guide wire.

FIG. 20 shows an aortic arch with an introducer catheter in place.

FIG. 21 shows an aortic arch with an introducer catheter in place and a percutaneous heart valve deployed after delivery through the introducer catheter.

FIG. 22 shows an alternate needle guide fixture configured for remote adjustment.

FIG. 23 shows an alternate needle guide fixture configured for remote adjustment with portions removed for better visualization of internal components.

### DETAILED DESCRIPTION OF INVENTION

The invention includes a system incorporating a guide wire and a deep access introducer device capable of measuring the proximity between the tip of the deep access introducer and the guide wire. More particularly, a system capable of characterizing both the proximity of the deep access introducer and the location along the guidewire of the deep access introducer is disclosed.

The system and components thereof can be used as stand alone units or in combination with a localizing guide wire and deep access introducer to compliment the use of a deep vascular access guide fixture. The guide fixture provides a path along which the deep access introducer, or other trocars, may be advanced and the methods of the present invention describe advantageous and synergistic use of each component described below. Additionally, the cooperation between the guide fixture and the deep access introducer facilitates alignment of a path or track with the target access site on the localizing guide wire.

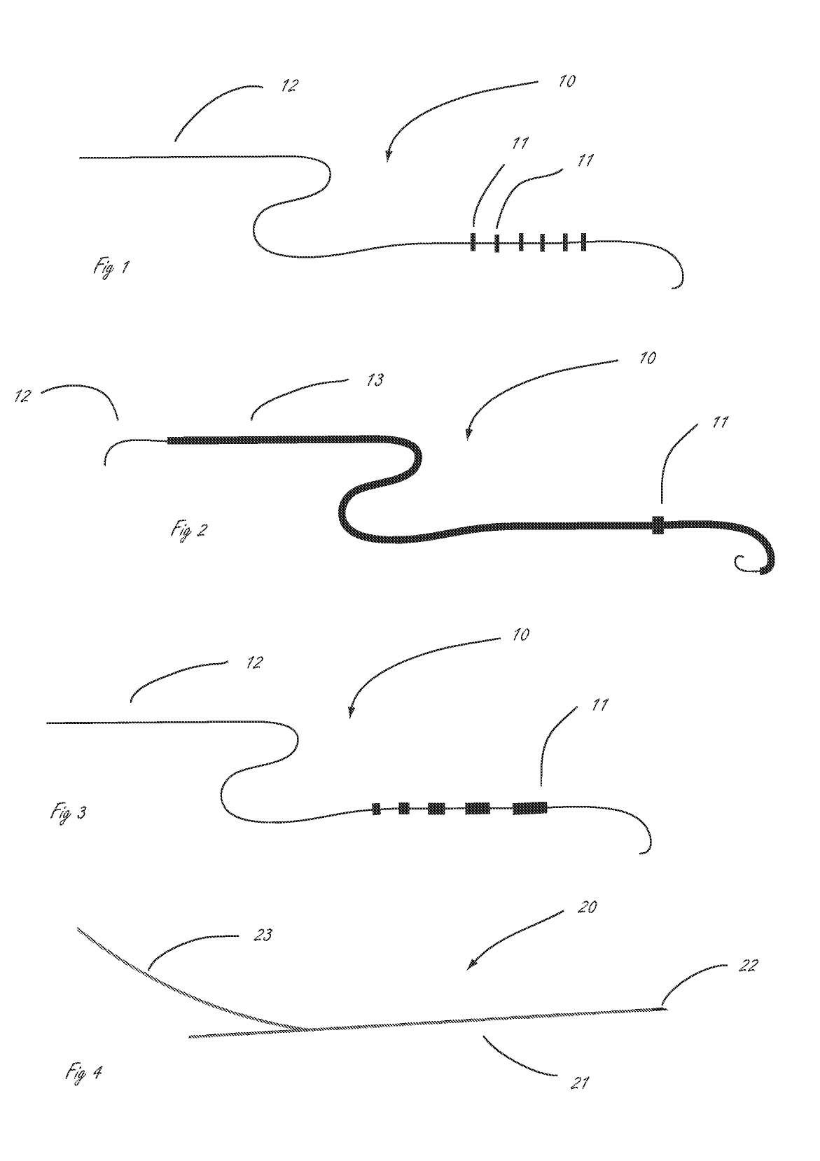

Referring generally to FIG. 1-3, a localizing guide wire means is described having an electrical or fluoroscopic guide wire 12, a movable element 13, and one or more locational elements 11.

Referring to FIG. 3, a fluoroscopic localizing guide wire 10 is comprised of guide wire 12 incorporating multiple locational elements 11 comprising segments of radio opaque material of differing lengths where the length of the segment identifies the relative position where along the length of the guide wire.

Referring to the multiple embodiments of FIG. 2, an electrical localizing guide wire 10 is disclosed comprising guide wire 12 incorporating a movable locational element 11 as part of a movable guide wire element 13, the locational element 11 has a low impedance surface. The locational element 11 provides visible detection under fluoroscopy where the locational element 11 is movable to a target location via a movable element 13 that rides on guide wire element 12 and a deep access introducer 20 that senses the impedance between the locational element 11 and its tip 22. By this combination, each of the locational element, the guide wire 12 and the introducer are localized both independently and relative to one another or where the guide fixture has 3 impedance sensors at 120 degrees and the three impedance measurements to element 11 define a set of parameters for setting the fixture. Impedance sensors as described herein may be either sources sinks or interchangeable, and any impedance system described herein incorporates at least one source.

Referring to FIG. 2, guide wire 12 incorporates a moveable locational element 11 comprising a fiber optic. The distal fiber optic tip is visible under fluoroscopy, such that the tip is movable to a target location under fluoroscopy movable element 13. Deep access introducer 20 comprising a tip 22 senses the optical output of the fiber optic and is localized relative to each other structure.

Referring to FIG. 2, a localizing guide wire 12 incorporates a movable locational element 11 comprising an ultrasonic transducer, is visible under fluoroscopy, and is movable on the guide wire via a movable element 13. The locational element 11 may additionally incorporate any combination of the following: the ability to characterize the mean cross sectional area of the surrounding vessel, the vessel wall thickness, aspects of the vessel wall compliance, and a deep access introducer incorporating an ultrasonic transducer capable of characterizing area, thickness, and the transducer's proximity to the ultrasonic transducer on the guide wire. The transducer may comprise a circular array. Different echos will indicate different boundaries associated with lumen and outer surface of vessel wall. Monitoring over time during a heart beat will provide information on vessel compliance.

Referring to FIG. 3, a modification to the embodiment of FIG. 1 provides a configuration wherein the guide wire 12 incorporates multiple stationary transducers along its length, each transducer signaling with a discrete and identifiable signal or pattern to distinguish each other and each transducer is additionally identifiable and distinct under fluoroscopy.

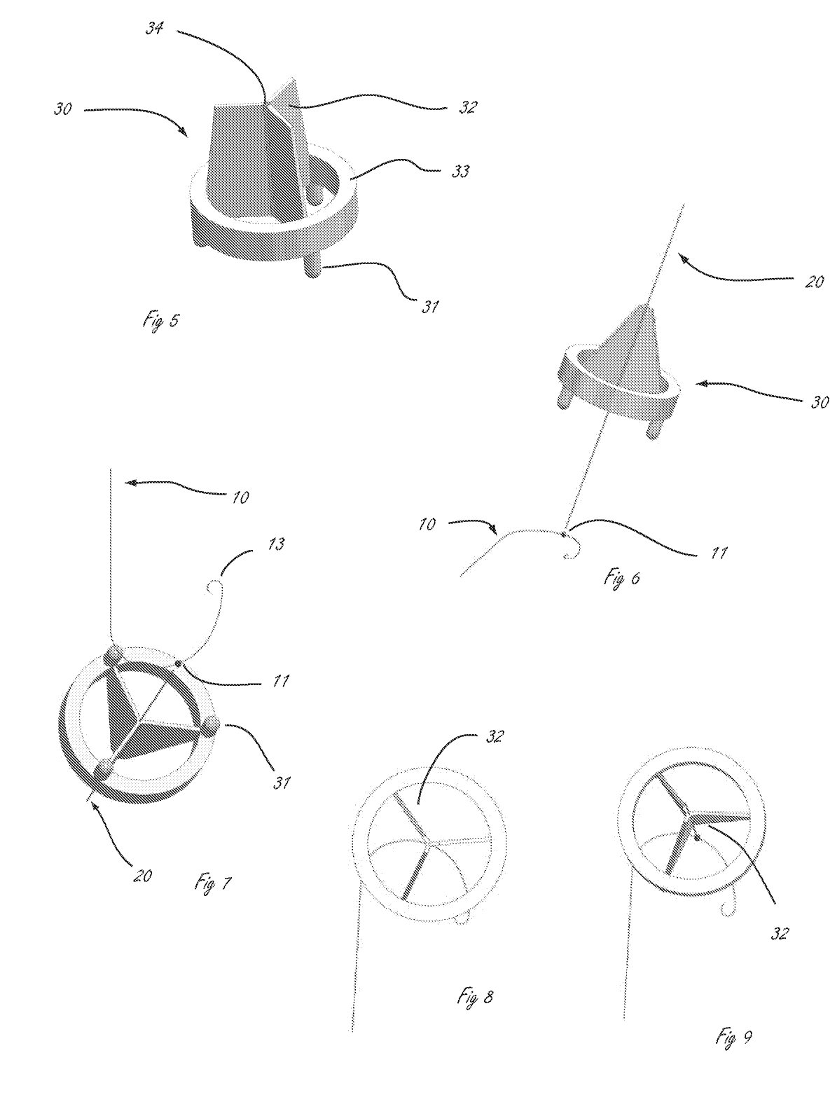

Referring to FIG. 4, a deep access introducer needle optionally comprises a radio opaque tip comprising a fixture 22 selected from the group consisting of an ultrasonic transducer, a magnet or a magnetic field sensing transducer, an electric field generating element, an impedance sensing transducer, a light source, and a light sensing transducer and combinations thereof.

Referring to FIGS. 5-9, a guide fixture 30 incorporates radio opaque target ring 33 and radio opaque target fins 32. Telescoping standoff legs 31 may comprise ultrasonic transducers or impedance sensors, and deep access introducer needle guidance path 34, which is aligned with the desired locational element 11 on the localizing guide wire 10. See FIG. 6. Alignment is achieved when the corresponding individual signals between the ultrasonic transducers comprised in the guide fixture and the locational element 11 and the-deep access introducer needle 20 are approximately equal. In an alternate embodiment impedance measurements may be used instead of ultrasonic signals. See FIG. 9.

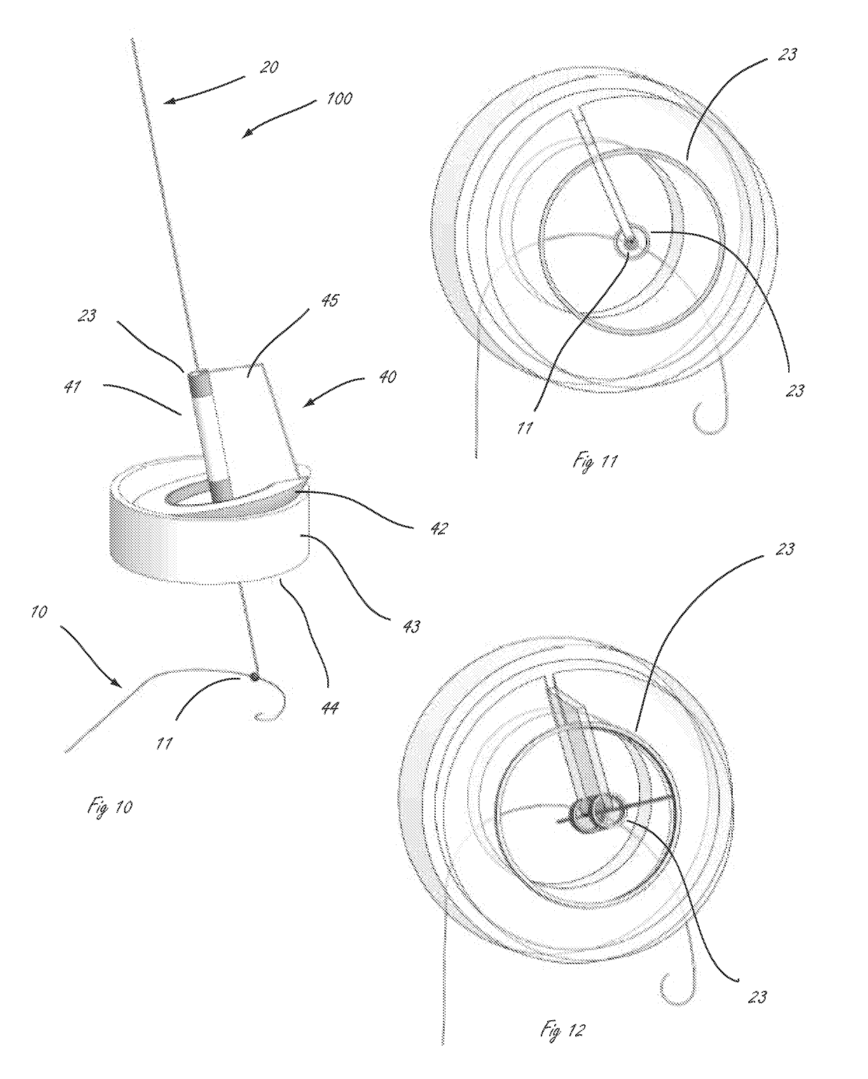

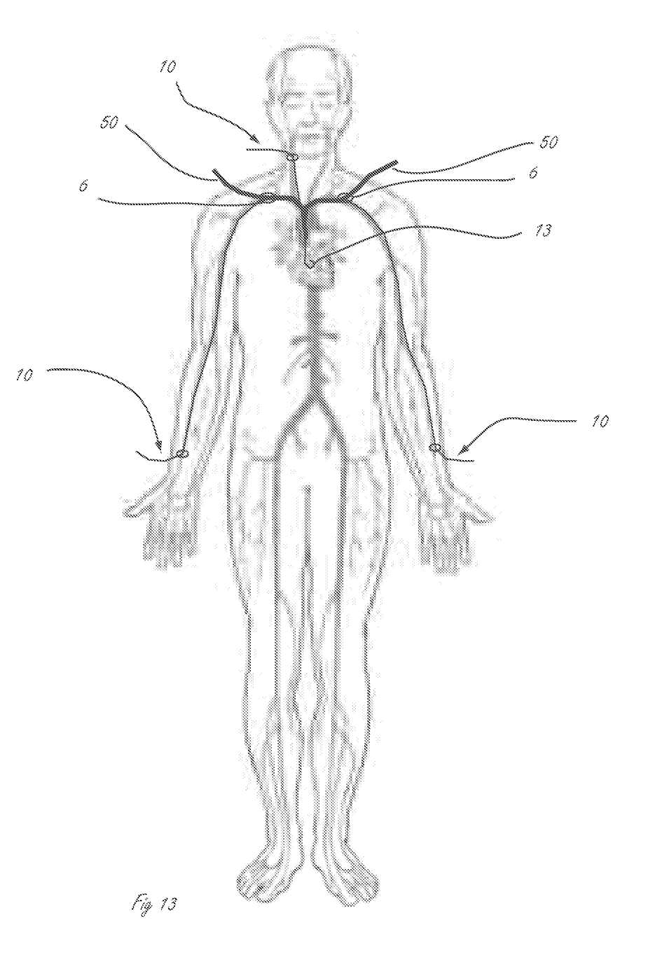

Referring to FIGS. 10-12, fluoroscopic guide fixture 40 comprises radio opaque target rings 23, a base 43, a rotational stage 42, and two piece deep access introducer needle guidance path comprised of the a channel created between removable guide half 41 and fixtured guide half 45. The removable guide half aids in the removal of the system after the deep access guide wire has been placed. Alignment is achieved when the locational element 11 is centered in target rings 23, typically by visual confirmation. On proper alignment, target ring images may additionally be circular as opposed to oval, and concentric. FIG. 11 illustrates an aligned guide while FIG. 12 illustrates a slightly misaligned guide. The target rings 23 are preferably circular, and in this configuration, appear oval when not properly aligned. The guide fixture 40 must be aligned with the focal plane of the fluoroscope. In general, all materials used in the fabrication of the fixture, except those used for the target rings are fabricated from materials with relatively low radioopacity. Rotational stage 42 and base 43 may be locked in place by drawing a vacuum at the interface surface (features not shown). In alternate designs the rotational stage may comprise a spherical section of greater then a hemisphere in which case the rotational stage may be clamped in place by arms extending from the base (not shown).

FIG. 13 indicates two common alternate entry sites for the introduction of the locational guide wire when the femorals are not compatable. These are the radial artery and carotid artery as indicated. As shown, the introducer 50 is then delivered via one of the brachial arteries.

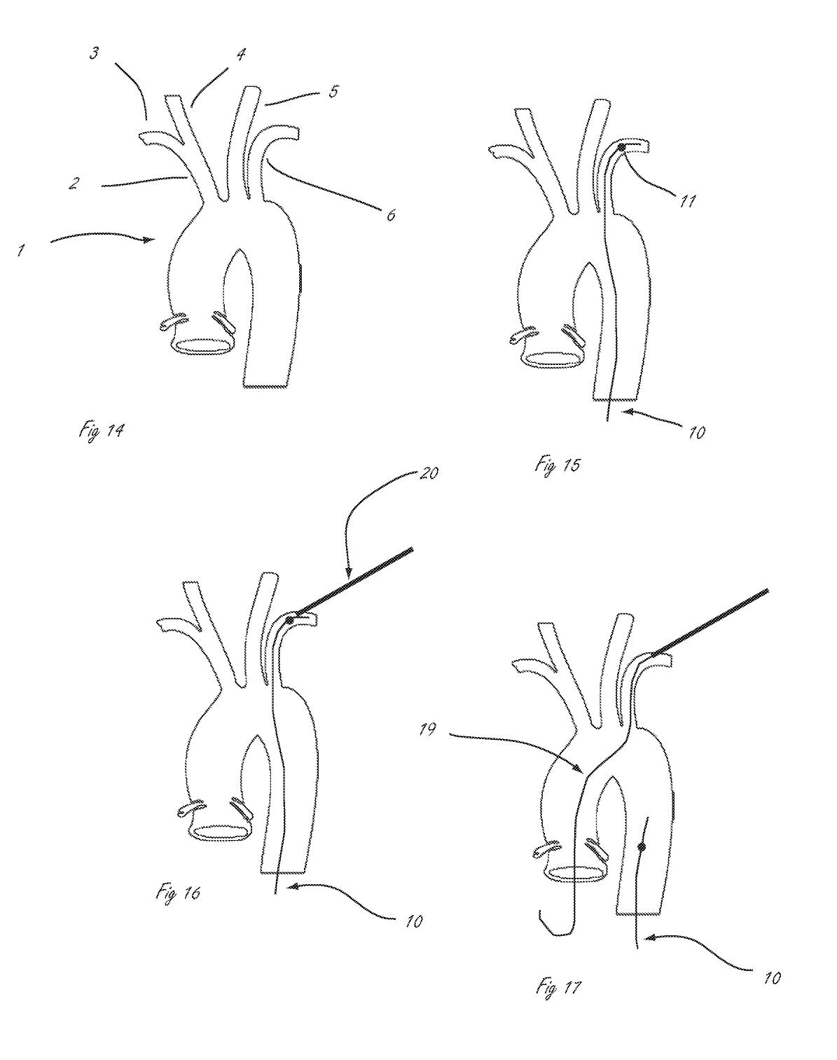

The practical use of the devices described above is as follows. Although, those at ordinary skill will appreciate that variances in the procedures will result from unique clinical circumstances addressed by the operator. Generally, the devices of the invention are applied in a percutaneous heart valve access procedure where deep vascular access is desired. The procedure as described uses a femoral approach for the localization catheter and a left subclavian artery access for the deep vascular access needle entry. This procedure is followed by the discrete steps that are specific for the ultrasonic and fluoroscopic based embodiments. Key points in the procedure are illustrated in FIGS. 14 through 20. FIG. 14 illustrates the aortic arch 1 and the branches emanating from the arch; the brachiocephalic artery 2, the right subclavian 3, the right common carotid 4, the left common carotid artery 5, and the left subclavian 6.

A localizing guide wire is introduced at the femoral artery and tracked through the aorta into the aortic arch 1 and then in to the left subclavian artery 6. Referring to FIG. 15, the localizing guide catheter is tracked, under fluoroscopy, to a point where the locational element 11 is situated in a portion of the left subclavian artery 6 of sufficient diameter to support the introduction of the percutaneously delivered valve. The vessel diameter can be characterized by introduction of an appropriate contrast agent and monitored by the distal ultrasonic transducer when using the ultrasonic embodiment of the localizing guide wire 10.

Once an appropriate location has been identified, the diameter of the vessel can be verified by fluoroscopically using the appropriate contrast agent. When using the configuration of FIG. 2 the contrast agent may be delivered via the lumen between the guide wire 12 and movable element 13. Placement and adjustment of the localizing guide fixture is now performed. Placement and adjustment are complete when the guide path is aligned with localizing element 11 and the system is vacuum locked in place as described herein elsewhere.

The deep access introducer needle 20 is then advanced through the center of the localizing guide fixture, through the patients intervening tissue, towards the locational element 11 on the localizing guide catheter as illustrated in FIG. 16. When present, transducers 22 on the distal end of the deep access introducer needles and comprised in the locational element 11 can be used to delineate proximity to the target site. The deep access introducer needle may comprise a relatively blunt tip which will tend to separate tissues at natural boundarys or may be sharp for piercing tissues.

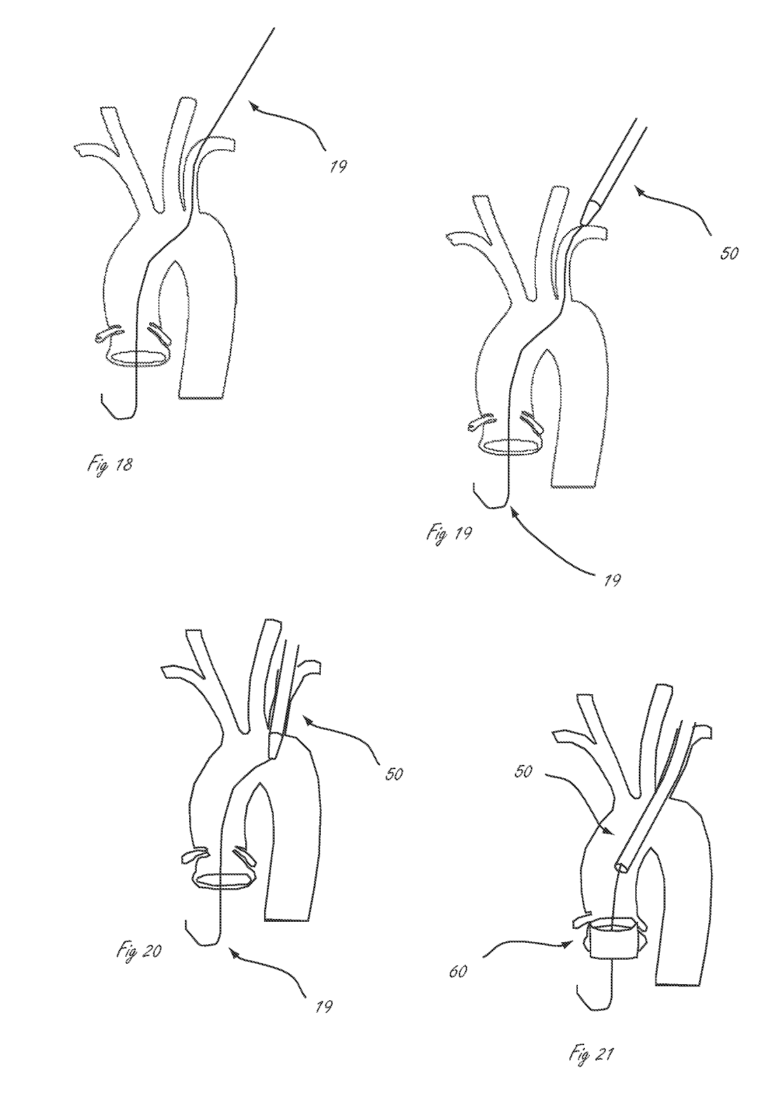

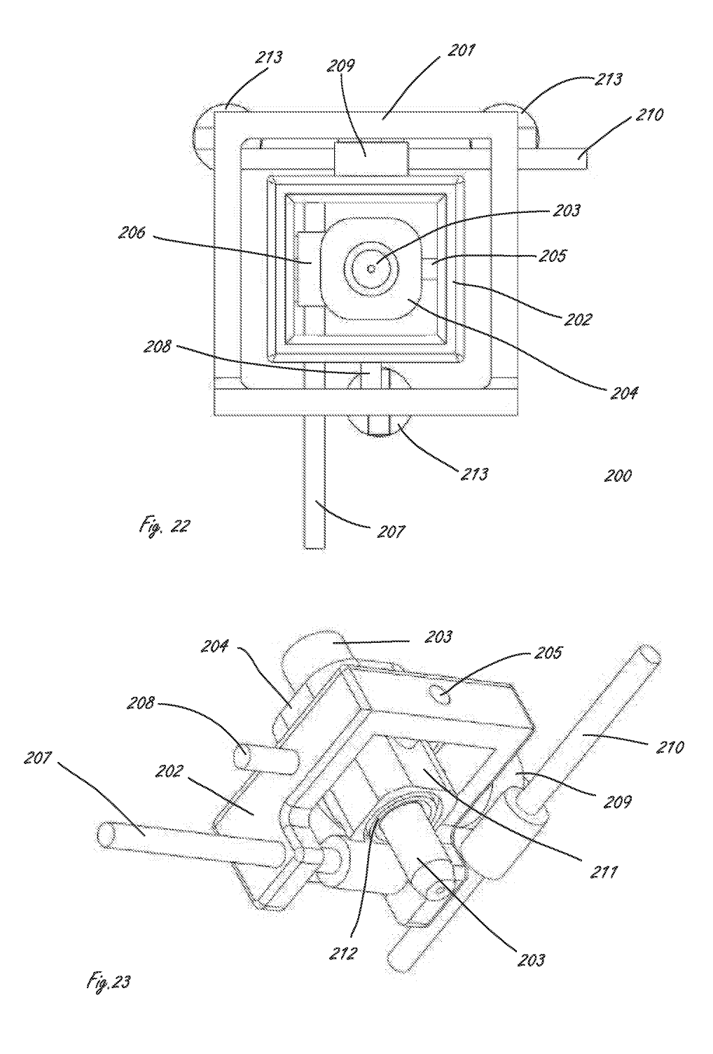

Once the distal end of the deep access introducer needle 20 has been placed within the target vessel at the target location, the localizing guide wire is fully or partially removed. The deep vascular access guide wire 19 is then placed as is illustrated in FIG. 17. Both the guidance system and the localizing guide wire are then completely removed from the operating field, leaving only the deep vascular access guide wire in place as is illustrated in FIG. 18. An introducer 50 along with its dilator is then delivered along the deep vascular access guide wire 19 as depicted in FIG. 19. After the introducer is introduced into the aorta at the target location and tracked to a position adjacent to the aortic valve, the dilator is removed as depicted in FIG. 20. The percutaneous heart valve replacement procedure then proceeds and the percutaneously delivered heart valve 60 is eventually placed as illustrated in FIG. 21.

Referring to FIGS. 10, 11 and 12, the system may be used under Flouroscopic guide as follows. Localizing guide fixture 40 is placed over the target site, with appropriate tissue access, on the patient and the bottom face 44 of the base 43 is affixed to the patient by a suitable means. Suitable means may be adhesive, vacuum, clamping, or other. The fluoroscope is then aligned such that the target site, and locational element 11 is centered within the fluoroscopic field of view. The alignment will also be such that a normal to the image plane will align with the intended trajectory of the deep access introducer needle.

The rotational stage 42 is then adjusted until the target rings are aligned as illustrated in FIG. 11. The locational element 11 may or may not be centered within the target rings, as illustrated in FIG. 11, at this time. If the locational element is not centered in the target rings as in FIG. 12, the rotational stage is adjusted appropriately and then the fluoroscope realigned to the new conformation. This can be an iterative process. Or alternatively the target rings may be aligned to correspond to the fluoroscopic conformation. Alignment is achieved when the locational element 11 is seen to be centered in target rings 23, target ring images. FIG. 11 illustrates an aligned guide while FIG. 12 illustrates a slightly misaligned guide.

Similarly, the system may be used under ultrasonic guide as follows. Localizing guide fixture 30 is placed over an ultrasonic coupling ring (not shown) over the target site, with appropriate tissue access, on the patient, and adjusted until the distance between each receiver leg 21 and the distal localizing transducer is equal. Under one transducer control protocol, these path lengths will be approximately equal when the time of flight for the signal arising from the distal localizing transducer on the localizing guide wire is equal for all receiver legs. Additional protocols will be obvious to those skilled in the art.

The localizing guide fixture 30 may be locked in place at this point. Locking in place can be achieved either by affixing the guide fixture to the patient using adhesive or to the table upon which the patient is laying. Alternatively a system similar to that of system 40 includes 3 transducers at 120 degree centers on its bottom interface. The distance between each of these transducers and the distal localizing transducer is measured and converted into a set of settings for the rotational stage 42, i.e. a rotational angle around the axis normal to the plane containing the three transducers comprised in or on the base and an angle relative to an axis normal to that plane.

The deep access introducer needle 20 may also be placed ultrasonically by guidance over a location with appropriate tissue access to the target site as indicated by fluoroscopic image. The deep access introducer needle 20 is then directed and advanced towards the locational element on the localizing guide wire by the physician. The physician uses feedback indicative of the proximity of the deep access introducer needle's tip 22 to the locational element to adjust the path of the deep access introducer needle 20 as it is advanced to the target site. Feedback is provided by the interaction between the two.

The deep access guide wire may be used with any of the present introducer sheath such as those manufactured by Sutura®, Vascular Solutions, Medtronics®, St. Jude®, Medafor® Inc., Cardiva Medical®, Access Closure™, or Abbot Vascular®.

FIGS. 22 and 23 illustrate a needle guide fixture configured for remote adjustment. The central hub 211 is generally of any conventional shape from cylindrical to rectangular parallelepiped shape. The longer dimension is perforated as to accommodate the needle holder 203. Both extremities of the longer dimension of the central hub are grooved 212 to accommodate radio opaque markers. The central hub 211 is connected to the middle frame 202 by means of a connecting inner shaft 205 in such a way that the central hub 211 can oscillate around the inner shaft 205. Also connected to the inner shaft 205 is the inner gear 6 which is bonded to the central hub 211. By actuating on the inner worm and screw shaft 207, the inner gear 206 is actuated and the central hub 211 oscillates around the inner shaft 205.

In similar fashion the middle frame 202 is connected to the outer frame 201 by means of the outer shaft 208 and outer gear 209. By actuating on the outer worm screw and shaft 210, the outer gear 209 is actuated and the middle frame 202 oscillates in relation to the outer frame 201 and around the outer shaft 208.

The outer frame feet 213 are integral part of the outer frame 201. By oscillating the middle frame 202 in relation to the outer frame 201, and the central hub 211 in relation to the middle frame 202, the operator changes the angle of the needle holder 203 in relation to the plane defined by the outer frame feet 213. The radio opaque marker grooves 212 provide a means to align the central hub 211 and orient the needle holder 203 to a pre-positioned marker in the relevant anatomy.

The procedure steps are as follows: The operator connects the assembly to the patient by means of the outer frame feet 213. These may be equipped with magnets that would connect to magnets pre-placed on the skin of the patient. The fixture may alternatively be affixed with adhesives directly to the patient, with surgical tape, or to the operating table as with other guide fixtures described herein. A target radio opaque marker target (not shown) is placed in the relevant anatomy. This could be a radio opaque guidewire inserted through a distant vessel. The fluorsoscope is generally placed in line with the needle holder 3 and the radio-opaque target (not shown). The operator actuates the worm shafts 207 and 210 to oscillate or tilt the middle frame 202 and the central hub 211 in relation to the outer frame 201 and the patient. When alignment is achieved between the radio opaque markers 212 and radio opaque target (not shown), the operator inserts a needle (not shown) through the needle holder 203 and incrementally advances the needle into the patient until the needle approaches the radio opaque marker and proper positioning is verified. An additional guidewire (not shown) is inserted through the needle and into the same vessel as the radio opaque target. The needle and needle holder 203 are then removed leaving the guidewire in place. The cap 4 is advanced through the guidewire. The cap 204 provides means of facilitating the introduction of a dilator through the central opening of the central hub 211. Whether the opening was previously occupied by the needle holder 203. The dilator and introducer assembly (not shown) is introduced over the guidewire and throughout the central hub 211 and into the vessel in the patient.

We claim:

1. A system for deep vascular access comprising.
a localizing guide wire;
a vascular access guide wire;
an introducer needle; and
a localizing needle guide fixture.

2. The system of claim 1 wherein the localizing guide wire has a locational element positioned along a length thereof.

3. The system of claim 2 wherein the localizing guide wire is comprised of a plurality of locational elements positioned along the length thereof.

4. The system of class 3 element of least 2 of the plurality of locational elements are visually distinct under fluoroscopy.

5. The system of class 1 wherein the localizing guide with further comprises an ultrasonic transducer.

6. The system of claim 5 wherein the localizing guide wire comprises a plurality of ultrasonic transducers, at least two of which are visibly distinct under fluoroscopy .

7. The system of claim 1 wherein the localizing guide wire is further comprises a fiber optic.

8. The system of claim 1 wherein the introducer needle has a radio opaque tip.

9. The system of claim 8 wherein the needle tip is comprised of a fixture selected from the group consisting of an ultrasonic transducer, a magnet, a magnetic field sensing transducer, an electric field generator, an impedance causing transducer, a light source, a light sensing transducer and combination thereof.

10. The system of claim 1 wherein the needle guide fixture has a circumference and a centrally disposed path therein.

11. The system of claim 1 wherein the needle guide fixture is comprised of a radio opaque structuring selected from the group consisting of a leg, a ring, a fin and pluralities and combinations thereof.

12. The system of claim 1 wherein the needle guide fixture is comprised of a base, at least two rings and a guidance path.

13. The system of claim 1 wherein the needle guide fixture is remotely adjustable and comprised of an inner shaft surrounded by a central hub.

14. The system of claim 13 wherein the needle guide is further comprised of an outer shaft operably connected to an outer gear.

15. The system of claim 1 wherein each of the localizing guide wire, the remote access guide wire, the introducer needle, and the needle guide fixture are visible under fluoroscopy.

16. The system of claim 1 further comprising a vessel dilator.

17. The system of claim 1 wherein the needle guide fixture has at least three impedance sensors spaced apart in an array.

18. A method for percutaneous and deep vascular access comprising establishing vascular access for a localizing guide wire (or catheter) and establishing vascular access for an introducer needle;
tracking the localizing guide wire into the aortic arch until a locational element affixed to the localizing guide wire is situated in the left subclavian artery.
placing a localizing needle guide fixture by alignment with the locational element;
advancing an introducer needle to traverse the localizing needle guide fixture, and into proximity with the locational element of the localizing guide catheter
at least partially removing the localizing guidewire;
placing the vascular access guide wire at the target site; and
delivering an introducer along the vascular access guide wire and adjacent to the aortic valve.

19. The method of claim 18 further comprising a dilation along with the introducer

20. The method of claim 18 further comprising the step of percutaneously replacing a heart valve.

21. The method of claim 18 wherein the step of tracking the localizing guide wire in to the aortic arch comprises infusing a contrast agent.

22. The method of claim 18 wherein the step of tracking the localizing guide wire into the aortic arch comprises activating an ultrasound transducer.

23. The method of claim 18 wherein the step of advancing the introducer needle to traverse the localizing needle guide fixture comprises passing the introducer needle through the center of the localizing needle guide fixture.

24. The method of claim 18 wherein the step of advancing the introducer needle into proximity with the locational element includes activating a transducer or the distal end of the needle.

25. The method of claim 18 wherein the vascular access for the localizing guide wire is femoral.

26. The method of clam 18 wherein the vascular access for the introducer needle is left subclavian.
